(19) United States
(12) Patent Application Publication
(10) Pub. No.: US 2025/0360242 A1
(43) Pub. Date: Nov. 27, 2025

(54) FRAGRANCE DIFFUSER

(71) Applicant: Guangdong Chiyang Scent Technology Co., LTD., Foshan (CN)

(72) Inventors: Xiaoyang GAO, Foshan (CN); Jiawen XU, Foshan (CN)

(21) Appl. No.: 19/083,725

(22) Filed: Mar. 19, 2025

(30) Foreign Application Priority Data
May 24, 2024 (CN) 202410651747.7
Dec. 30, 2024 (CN) 202423281826.8

(51) Int. Cl. A61L 9/14

(52) U.S. Cl. CPC A61L 9/145; A61L 2209/133; A61L 2209/134

(57) ABSTRACT

A fragrance diffuser includes a housing and a bottle defining an accommodation cavity therein. An atomizing head assembly is disposed in the housing and connected to the bottle. The atomizing head assembly includes a liquid-conducting core rod, an atomizing sheet, an atomizing seat and a casing pipe. The atomizing seat is connected to the bottle and defines a liquid accumulation space connected to the accommodation cavity and located below the accommodation cavity. The casing pipe is located in the atomizing seat, the housing defines a mist outlet, and the casing pipe is located on a side of the atomizing sheet facing away from the mist outlet. The liquid-conducting core rod is inserted into the casing pipe, a first end of the liquid-conducting core rod is connected to the liquid accumulation space, and a second end of the liquid-conducting core rod is abutted against the atomizing sheet.

### CROSS-REFERENCE TO RELATED APPLICATION

This application claims priority to Chinese Patent Application No. 202410651747.7, filed on May 24, 2024, which is herein incorporated by reference in its entirety. This application also claims priority to Chinese Patent Application No. 202423281826.8, filed on Dec. 30, 2024, which is herein incorporated by reference in its entirety.

### TECHNICAL FIELD

The disclosure relates to the technical field of fragrance devices, and more particularly to a fragrance diffuser.

### BACKGROUND

Fragrance device is an electrical appliance that atomizes liquid fresheners such as fragrance or essential oils and discharges them into the air to diffuse the aroma of the fresheners into the air to improve air quality, which has been widely used in various occasions such as homes, hotel rooms, lobbies, corridors, and cars.

In the related art, atomization methods of the fragrance device are mainly divided into ultrasonic atomization and pneumatic atomization. The ultrasonic atomization method is more widely used due to its simple structure and easy automatic control. The ultrasonic atomization method is to disperse liquid fragrance (essential oil) into small particles (atomization) through high-frequency vibration of an ultrasonic atomizing component, and the liquid fragrance with small particles enters the space environment through micropores of the atomizing component.

In the related art, there are inverted ultrasonic atomization fragrance devices, which usually require a bottle containing liquid fragrance to be inverted, so that the liquid fragrance can flow by its own gravity and then be atomized. However, in this way, the liquid fragrance often leaks significantly at a mist outlet, which not only makes an outer surface of the fragrance device easy to form sludge and become dirty, causing inconvenience in use, but also causes waste of the liquid fragrance.

### SUMMARY

In view of this, an objective of the disclosure is to provide a fragrance diffuser (also referred to as aroma diffuser) to solve a problem that atomized particles are easy to fall on a surface of an existing ultrasonic atomizing fragrance device by adopting a top mist outlet mode.

In order to solve the above technical problem, the technical solution used by the disclosure is as follows.

The disclosure provides a fragrance diffuser, including a housing and a bottle defining an accommodation cavity therein. An atomizing head assembly is disposed in the housing and connected to the bottle. The atomizing head assembly includes a liquid-conducting core rod, an atomizing sheet, an atomizing seat and a casing pipe. The atomizing seat is connected to the bottle and defines a liquid accumulation space therein, and the liquid accumulation space is connected to the accommodation cavity and located below the accommodation cavity. The casing pipe is located in the atomizing seat, the housing defines a mist outlet facing towards the atomizing sheet, and the casing pipe is located on a side of the atomizing sheet facing away from the mist outlet. The liquid-conducting core rod is inserted into the casing pipe, a first end of the liquid-conducting core rod is connected to the liquid accumulation space, a second end of the liquid-conducting core rod is abutted against the atomizing sheet, the liquid-conducting core rod is configured to flow liquid fragrance in the liquid accumulation space from the first end of the liquid-conducting core rod to the second end of the liquid-conducting core rod, and the liquid fragrance is atomized by the atomizing sheet and then disperses into air from the mist outlet.

In an embodiment, a first divider is disposed in the atomizing seat and located above the casing pipe, and the first divider is configured to divide an upper cavity and a lower cavity in the atomizing seat. The upper cavity is connected to the lower cavity, and the casing pipe and the liquid-conducting core rod are located in the lower cavity.

In an embodiment, a second divider is disposed on a peripheral surface of the casing pipe and extending radially along the casing pipe, and a peripheral side of the second divider is connected to the first divider and an inner wall of the atomizing seat to divide the lower cavity into a first cavity and a second cavity. The first divider defines a first through-hole, the second divider defines a second through-hole, the first through-hole is configured to connect the upper cavity and the lower cavity, and the second through-hole is configured to connect the first cavity and the second cavity.

In an embodiment, the first divider further defines a third through-hole, the first through-hole and the third through-hole are respectively located at two sides of the second divider, and the third through-hole is configured to connect the upper cavity and the second cavity.

In an embodiment, the second through-hole is one in quantity, and the second through-hole is located at a lower half of the second divider; the second through-hole is multiple in quantity, and the multiple second through-holes are arranged on the lower half of the second divider at intervals; or the second through-hole is three in quantity, and one of the three second through-holes is located at a middle position of a bottom of the second divider, and the other two of the three second through-holes are located on a middle part of the second divider and respectively located at two sides of the liquid-conducting core rod.

In an embodiment, a diameter of the second through-hole is not less than 1 millimeter (mm), and a diameter of the third through-hole is not less than 1.5 mm.

In an embodiment, a third divider is disposed on a peripheral surface of the casing pipe between the atomizing seat and the second divider and extending radially along the casing pipe. A peripheral side of the third divider is connected to the inner wall of the atomizing seat to define a cavity A between the third divider and the second divider, and define a cavity B between the third divider and the atomizing sheet, and the cavity A is separated from the cavity B. The first divider further defines a third through-hole, and the third through-hole is configured to connect the upper cavity and the cavity A.

In an embodiment, the atomizing seat includes a body and a mist outlet head detachably connected to the body. The casing pipe is disposed in the body, and the atomizing sheet is fixed between the body and the mist outlet head. The mist outlet head defines a mist outlet channel, an end of the mist outlet channel is disposed facing towards the atomizing sheet, and another end of the mist outlet channel is butted with the mist outlet.

In an embodiment, a first groove is defined between the body and the mist outlet in an annular configuration, and a sealing ring is disposed in the first groove. The sealing ring defines a second groove, and a peripheral edge of the atomizing sheet is embedded in the second groove.

In an embodiment, the first groove is an annular U-shaped groove, the sealing ring is U-shaped and matched with the first groove, and outer side surfaces of the sealing ring are configured to tightly fit with corresponding surfaces of the first groove respectively. The sealing ring defines a first notch, the atomizing seat defines a second notch, and a power cord connected to the atomizing sheet sequentially passes through the first notch and the second notch and winds out of the atomizing seat.

In an embodiment, a base plate is disposed in the housing, a power supply is disposed on a side of the base plate, and a controller is disposed on another side of the base plate. The power supply and the controller are detachably connected to the base plate, the power supply is electrically connected to the controller, the atomizing sheet is electrically connected to the controller, and the base plate is connected to the body.

In an embodiment, the housing includes an upper housing, a lower housing and a connecting housing. The connecting housing is fixedly sleeved on a periphery of the body, and the upper housing covers the bottle and is clamped with an upper end of the connecting housing.

The lower housing includes a first housing part, a second housing part and a third housing part. The third housing part defines the mist outlet, the third housing part is matched with the controller, and the mist outlet is butted with the mist outlet channel of the mist outlet head after the third housing part is matched with the controller.

The first housing part and the second housing part are spliced and connected to cover the connecting housing, the power supply and the controller and fixedly clamp the third housing part.

In an embodiment, the atomizing head assembly further includes a pressure relief mechanism, and the accommodation cavity is connected to the liquid accumulation space through the pressure relief mechanism.

In an embodiment, a first divider is disposed in the atomizing seat and located above the casing pipe, and the first divider is configured to divide a pressure relief cavity and a lower cavity in the atomizing seat. The pressure relief cavity is connected to the lower cavity, and the casing pipe and the liquid-conducting core rod are located in the lower cavity. The pressure relief mechanism is detachably installed in the pressure relief cavity, and the accommodation cavity is connected to the pressure relief cavity through the pressure relief mechanism.

In an embodiment, the pressure relief mechanism defines a pressure relief hole and a gas return hole, an input end and an output end of the gas return hole are respectively connected to the pressure relief cavity and the accommodation cavity, and an input end and an output end of the pressure relief hole are respectively connected to the accommodation cavity and the pressure relief cavity.

In an embodiment, the gas return hole is higher than the pressure relief hole, and a cross-section of the pressure relief hole is greater than that of the gas return hole.

The pressure relief mechanism defines an oil accumulation groove, and the oil accumulation groove is located on a bottom of the accommodation cavity and connected to the accommodation cavity. The pressure relief hole is defined on a bottom of the oil accumulation groove, and the gas return hole is higher than the oil accumulation groove.

The output end of the pressure relief hole defines a liquid-conducting groove connected to the pressure relief hole, and a sectional area of the liquid-conducting groove is greater than that of the pressure relief hole. The liquid-conducting groove is disposed extending along the pressure relief cavity, and an output end of the liquid-conducting groove is connected to the pressure relief cavity.

The input end of the gas return hole defines a gas accumulation groove connected to the gas return hole, and a sectional area of the gas accumulation groove is greater than that of the gas return hole. The gas accumulation groove is disposed extending along the pressure relief cavity, and an input end of the gas accumulation groove is connected to the pressure relief cavity.

In an embodiment, the first divider further defines a first through-hole and a third through-hole, the first through-hole and the third through-hole are respectively located on two sides of the second divider, and the first through-hole and the third through-hole are configured to connect the pressure relief cavity and a second cavity. Positions of the first through-hole and the third through-hole are respectively configured to correspond to the pressure relief hole and the gas return hole.

An end of the pressure relief cavity facing away from the first divider defines an opening, an end of the pressure relief mechanism is inserted into the opening, and the pressure relief mechanism is in sealing fit with the opening. The pressure relief mechanism includes a limit flange at the opening, and the limit flange protrudes outward from the opening. A bottom of the bottle has a bottle mouth connected to the accommodation cavity, the gas return hole is defined on the bottle mouth and extends along a direction of the accommodation cavity, and the pressure relief hole is located below the bottle mouth.

In an embodiment, the atomizing head assembly further includes a middle element, and the middle element is disposed between the second end of the liquid-conducting core rod and the atomizing sheet. A side of the middle element is connected to the second end of the liquid-conducting core rod, and another side of the middle element is connected to the atomizing sheet. The middle element is configured to transport the liquid fragrance from the second end of the liquid-conducting core rod to the atomizing sheet, a concentration of the liquid fragrance on the atomizing sheet is lower than a concentration of the liquid fragrance on the second end of the liquid-conducting core rod, and the atomizing sheet is configured to atomize the liquid fragrance on the middle element and disperse atomized liquid fragrance into air from the mist outlet.

In an embodiment, a connection area of the middle element and the liquid-conducting core rod is smaller than that of the middle element and the atomizing sheet. The middle element is a liquid-conducting cotton sheet.

Compared to the related art, the beneficial effects of the fragrance diffuser described by the disclosure are mainly reflected in the follows.

In the fragrance diffuser of the disclosure, the atomizing seat has the liquid accumulation space, the casing pipe is disposed in the atomizing seat, the first end of the liquid-conducting core rod is connected to the liquid accumulation space, and the second end of the liquid-conducting core rod is abutted against the atomizing sheet. The liquid-conducting core rod is inserted into the casing pipe, which can reduce liquid pressure on the surface of the liquid-conducting core rod, thereby reducing the liquid fragrance flowing to the second end of the liquid-conducting core rod, and avoiding excessive liquid fragrance flowing to the second end of the liquid guiding core rod, so that the liquid fragrance in the liquid accumulation space is not easy to leak out of the mist outlet, thereby reducing leakage.

### BRIEF DESCRIPTION OF DRAWINGS

The above and other objectives, features and advantages of the disclosure will become more clear by more specific description of embodiments of the disclosure shown in accompanying drawings. The same reference signs indicate the same parts in all the accompanying drawings, and the accompanying drawings are not deliberately scaled to the actual size, and the focus is on illustrating the main purpose of the disclosure.

FIG. 1 illustrates a schematic diagram from a front perspective of a fragrance diffuser according to an embodiment of the disclosure.

FIG. 2 illustrates a schematic sectional diagram of the fragrance diffuser according to an A-A line in FIG. 1.

FIG. 3 illustrates a schematic diagram from a left perspective of the fragrance diffuser according to the embodiment of the disclosure.

FIG. 4 illustrates a schematic sectional diagram of the fragrance diffuser according to a B-B line in FIG. 3.

FIG. 5 illustrates a schematic diagram of a part A in FIG. 2.

FIG. 6 illustrates a schematic diagram of a part B in FIG. 4.

FIG. 7 illustrates an internal view of the fragrance diffuser according to the embodiment of the disclosure.

FIG. 8 illustrates a structural exploded view of the fragrance diffuser according to the embodiment of the disclosure.

FIG. 9 illustrates a schematic sectional structural diagram of a fragrance diffuser according to another embodiment of the disclosure.

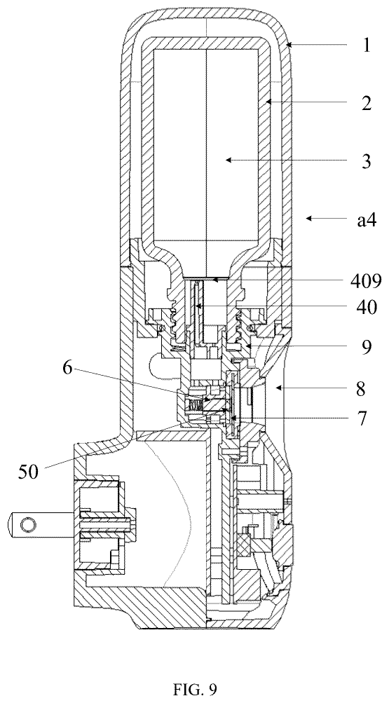

FIG. 10 illustrates a schematic partial enlarged structural diagram of FIG. 9.

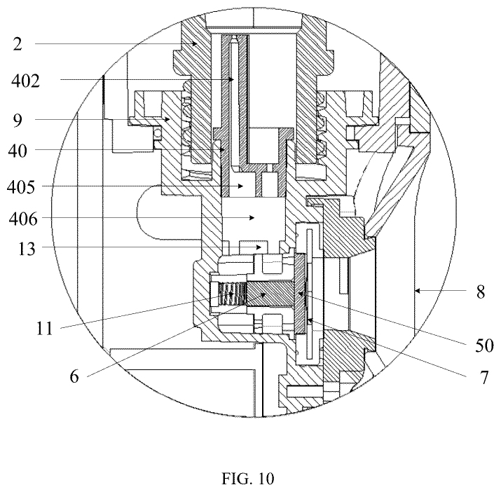

FIG. 11 illustrates a schematic structural diagram from another perspective of the fragrance diffuser according to the another embodiment of the disclosure.

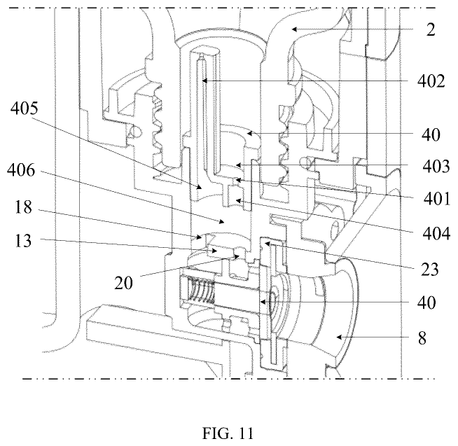

FIG. 12 illustrates a schematic structural diagram of a pressure relief mechanism of the fragrance diffuser according to the another embodiment of the disclosure.

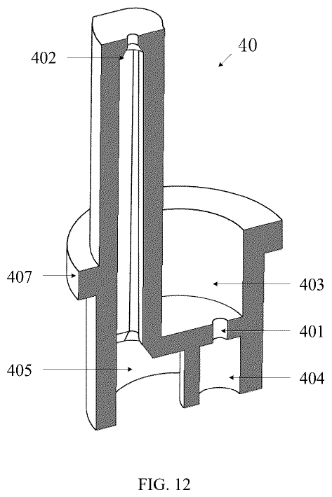

FIG. 13 illustrates a schematic structural diagram of assembly of an atomizing seat, a liquid-conducting core rod, a slow-release element and an atomizing sheet of the fragrance diffuser according to the another embodiment of the disclosure.

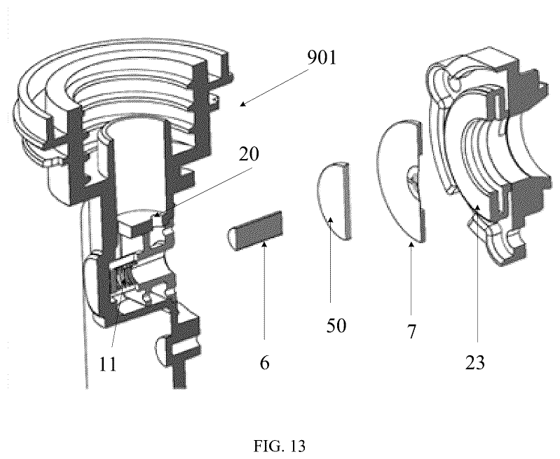

### DESCRIPTION OF REFERENCE SIGNS

1—housing; 2—bottle; 3—accommodation cavity; 4—power supply; 5—controller; 6—liquid-conducting core rod; 7—atomizing sheet; 8—mist outlet; 9—atomizing seat; 901—body; 902—mist outlet head; 903—mist outlet channel; 904—liquid accumulation space; 10—casing pipe; 11—elastic element; 12—opening; 13—first divider; 14—second divider; 15—first cavity; 16—second cavity; 161—cavity A; 162—cavity B; 17—upper cavity; 18—first through-hole; 19—second through-hole; 20—third through-hole; 21—third divider; 22—first groove; 23—sealing ring; 24—second groove; 25—first protruding part; 26—second protruding part; 27—third protruding part; 28—first notch; 29—second notch; 30—power cord; 31—base plate; 32—upper housing; 33—first housing part; 34—second housing part; 35—third housing part; 36—control button; 37—working status indicator lamp; 38—first housing; 39—second housing; 40—pressure relief mechanism; 401—pressure relief hole; 402—gas return hole; 403—oil accumulation groove; 404—liquid guide groove; 405—gas accumulation groove; 406—pressure relief cavity; 407—limit flange; 408—opening; 409—bottle mouth; 50—middle element; 60—connecting housing.

### DETAILED DESCRIPTION OF EMBODIMENTS

The technical solution of the disclosure is further described in detail below in conjunction with the accompanying drawings and specific embodiments, so that those skilled in the art can better understand the disclosure and implement it. However, the embodiments cited are not intended to limit the disclosure. In the embodiments, it should be understood that orientations or positional relationships indicated by terms such as “longitudinal”, “lateral”, “upper”, “lower”, “front”, “rear”, “left”, “right”, “vertical”, “horizontal”, “top”, “bottom”, “inside” and “outside” are based on the orientations or positional relationships shown in the accompanying drawings, and are merely for the convenience of describing the disclosure, rather than indicating or implying that the device or element referred to must have a specific orientation, be constructed and operated in a specific orientation, and therefore cannot be understood as a limitation of the disclosure.

It should be noted that when an element is considered to be “connected” to another element, it may be directly connected to the other element and integrated therewith, or there may be an intermediate element at the same time. The terms “installed”, “an end”, “another end” and similar expressions used in the disclosure are for illustrative purposes only.

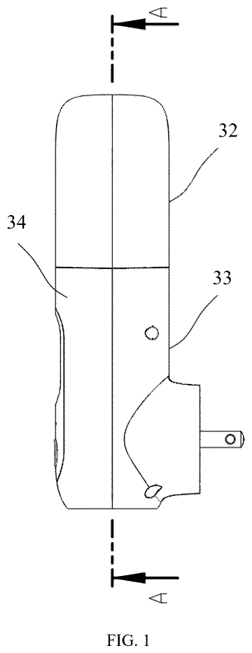

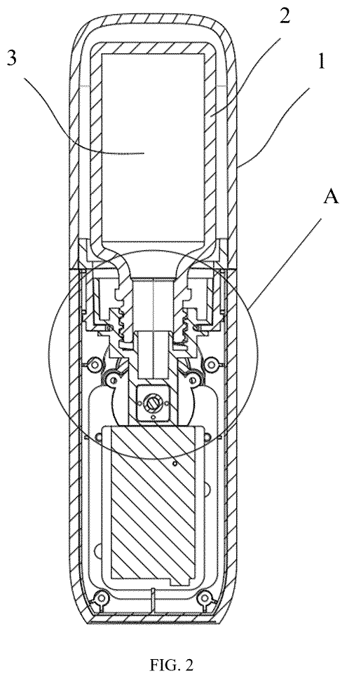

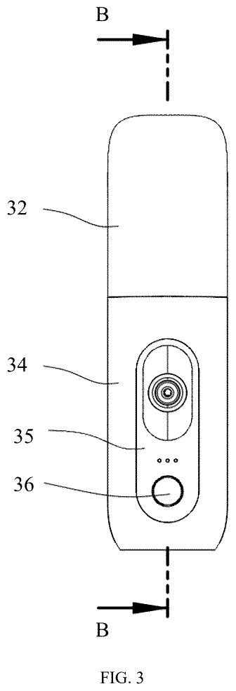

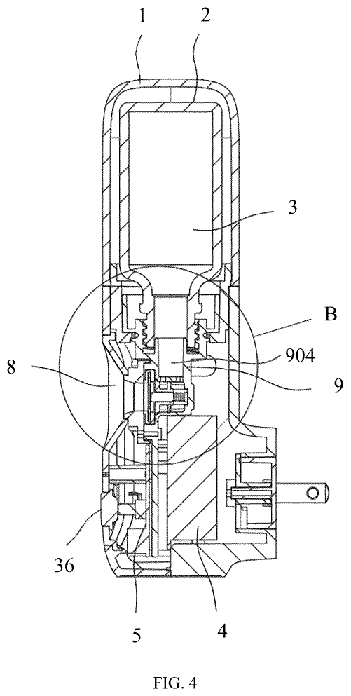

The embodiment provides a fragrance diffuser, as shown in FIGS. 1-8, the fragrance diffuser includes a housing 1 and a bottle 2 defining an accommodation cavity 3 therein. An atomizing head assembly is disposed in the housing 1 and connected to the bottle 2. The atomizing head assembly includes a liquid-conducting core rod 6, an atomizing sheet 7, an atomizing seat 9 and a casing pipe 10. The atomizing seat 9 is connected to the bottle 2, and defines a liquid accumulation space 904 connected to the accommodation cavity 3 and located below the accommodation cavity 3. The casing pipe 10 is located in the atomizing seat 9. The housing 1 defines a mist outlet 8 facing towards the atomizing sheet 7, and the casing pipe 10 is located on a side of the atomizing sheet 7 facing away from the mist outlet 8. The liquid-conducting core rod 6 is inserted into the casing pipe 10, a first end of the liquid-conducting core rod 6 is connected to the liquid accumulation space 904, and a second end of the liquid-conducting core rod 6 is abutted against the atomizing sheet 7. The liquid-conducting core rod 6 is configured to flow liquid fragrance in the liquid accumulation space 904 from the first end of the liquid-conducting core rod 6 to the second end of the liquid-conducting core rod 6, so that the liquid fragrance is atomized by the atomizing sheet 7, and then diffused to the air from the mist outlet 8. Specifically, the atomizing seat 9 and the casing pipe 10 can be an integral structure or a split connection structure.

In the fragrance diffuser of the disclosure, the atomizing seat 9 has the liquid accumulation space 904, the casing pipe 10 is disposed in the atomizing seat 9, the first end of the liquid-conducting core rod 6 is connected to the liquid accumulation space 904, and the second end of the liquid-conducting core rod 6 is abutted against the atomizing sheet 7. The liquid-conducting core rod 6 is inserted into the casing pipe 10, which can reduce liquid pressure on the surface of the liquid-conducting core rod 6, thereby reducing the liquid fragrance flowing to the second end of the liquid-conducting core rod 6, and avoiding excessive liquid fragrance flowing to the second end of the liquid guiding core rod 6, so that the liquid fragrance in the liquid accumulation space 904 is not easy to leak out of the mist outlet 8, thereby reducing leakage.

In an embodiment, a first divider 13 is disposed in the atomizing seat 9 and located above the casing pipe 10. The first divider 13 is configured to divide an upper cavity 17 and a lower cavity in the atomizing seat 9, that is, above the first divider 13 is the upper cavity 17, and below the first divider 13 is the lower cavity, and the upper cavity 17 is connected to the lower cavity. The casing pipe 10 and the liquid-conducting core rod 6 are located in the lower cavity. By setting the first divider 13 and setting the casing pipe 10 and the liquid-conducting core rod 6 in the lower cavity, the liquid pressure on the surface of the liquid-conducting core rod 6 is further reduced.

Specifically, a first end (i.e., an end of the casing pipe 10 facing away from the atomizing sheet 7) of the casing pipe 10 is embedded into an inner wall of the atomizing seat 9 with a preset depth, thereby fixing the casing pipe 10. An elastic element 11 is disposed in the casing pipe 10, an end of the elastic element 11 is abutted against the first end of the liquid-conducting core rod 6, and another end of the clastic element 11 is abutted against the first end of the casing pipe 10 or the inner wall of the atomizing seat 9 opposite to the first end of the casing pipe 10. The liquid-conducting cire rod 6 is slidably connected to the casing pipe 10, so that elastic deformation of the clastic element 11 can ensure that the liquid-conducting core rod 6 vibrates in coordination with high-frequency vibration of the atomizing sheet 7. A wall of the casing pipe 10 defines an opening 12, which allows the liquid fragrance to flow into the casing pipe 10. Certainly, in order to ensure stability of the casing pipe 10, a structure that can be embedded into the inner wall of the atomizing seat 9 can be also disposed on a second end (i.e., an end of the casing pipe 10 proximate to the atomizing sheet 7) of the casing pipe 10, so that a supporting force can be formed at both ends of the casing pipe 10, to thereby improve the stability of the casing pipe 10. In an embodiment, the opening 12 is located right above the clastic element 11 and extends axially to the first end of the liquid-conducting core rod 6, to thereby ensure filling and smoothness of the liquid fragrance at the first end of the liquid-conducting core rod 6. A radial gap between the liquid-conducting core rod 6 and the casing pipe 10 should not be too large, which can not only ensure the stability of the direction of the liquid-conducting core rod 6 during axial vibration, but also avoids filling the gap with too much liquid fragrance, which may cause resistance to the vibration of the liquid-conducting core rod 6 and lead to leakage. The gap is 0.1 mm.

In an embodiment, a second divider 14 is disposed on a peripheral surface of the casing pipe 10 and extending radially along the casing pipe 10, and a peripheral side of the second divider 14 is connected to the first divider 13 and the inner wall of the atomizing seat 9 to divide the lower cavity into a first cavity 15 and a second cavity 16. The first divider 13 defines a first through-hole 18, and the second divider 14 defines a second through-hole 19. The first through-hole 18 is configured to connect the upper cavity 17 and the first cavity 15, so that the liquid fragrance in the upper cavity 17 flows into the first cavity 18 through the first through-hole 18. The second through-hole 19 is configured to connect the first cavity 15 and the second cavity 16, so that the liquid fragrance can flow from the first cavity 15 to the second cavity 16.

In an embodiment, the first divider 13 further defines a third through-hole 20, the first through-hole 18 and the third through-hole 20 are respectively located on two ends of the second divider 14, and the third through-hole 20 is configured to connect the upper cavity 17 and the second cavity 16.

When the fragrance diffuser of the embodiment is in atomization work, a top of the accommodation cavity 3 has a cavity filled with air due to the inverted bottle 2, and the liquid fragrance is below the cavity. The liquid fragrance flows into the upper cavity 17 of the atomizing seat 9, and then flows into the first cavity 15 through the first through-hole 18. After passing through the wall of the casing pipe 10, the liquid fragrance contacts with the first end of the liquid-conducting core rod 6, and is attracted to the second end of the liquid-conducting core rod 6 for atomization. As the liquid fragrance in the first cavity 15 is continuously atomized, the liquid fragrance in the accommodation cavity 3 will also continuously flow into the first cavity 15 as a supplement, thereby forming a flow. The gas outside the fragrance diffuser will enter the atomizing seat 9 through the atomizing sheet 7 and the liquid-conducting core rod 6, and be mixed in the liquid fragrance. The gas mixed in the liquid fragrance will gradually move to the cavity above the accommodation cavity 3 to supplement the space of the consumed liquid fragrance and ensure that the cavity is at a positive pressure. In the embodiment, by setting the second divider 14, the second through-hole 19 and the third through-hole 20, the gas mixed in the liquid fragrance in the first cavity 15 can be promoted to enter the second cavity 16 through the second through-hole 19, enter the upper cavity 17 upward through the third through-hole 20, and then continue to move upward to the cavity, which can avoid formation of a counter-turbulence between upward-flowing gas and downward-flowing liquid fragrance, and make the flow of the gas and the flow of the liquid fragrance achieve a smooth internal circulation. It not only provides a smoother channel for the gas flow, but also provides conditions for the smooth flow of the liquid fragrance, so that a stable reflux effect can be formed, thereby ensuring continuity and stability of the atomization process. The second divider 14 also has a certain blocking effect, which can prevent the gas in the second cavity 16 and the liquid fragrance flowing from the upper cavity 17 into the first cavity 15 from mixing with each other, and can further promote the formation of the stable reflux. In an embodiment, a peripheral side of the second divider 14 is tightly matched with the first divider 13 and the inner wall of the atomizing seat 9.

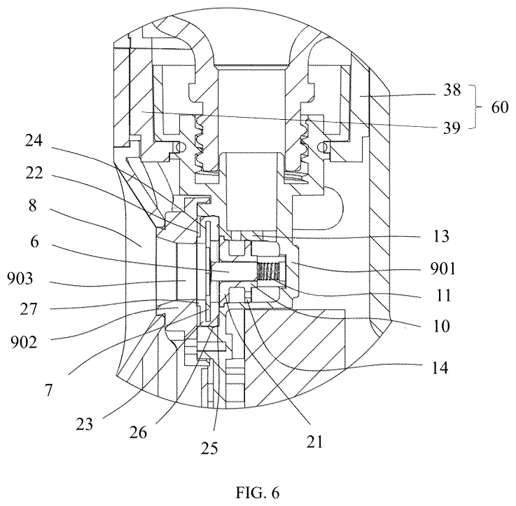

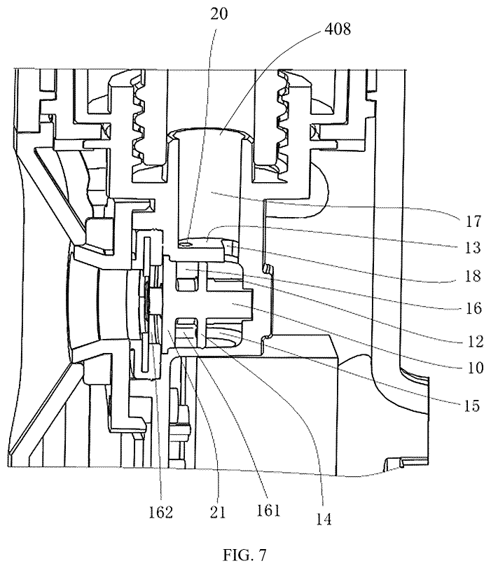

As described above, the first through-hole 18 and the third through-hole 20 need to have preset interval distances to avoid convection. In order to ensure sufficient fluidity of the liquid fragrance, an opening size of the first through-hole 18 is usually greater than that of the third through-hole 20. In an embodiment, as shown in FIG. 6 and FIG. 7, the first through-hole 18 extends inward from an edge of the first divider 13, thereby increasing the opening size of the first through-hole 18. In order to ensure smooth airflow backflow, the third through-hole 20 is located right above the second cavity 16, and a diameter of the third through-hole 20 is not less than 1.5 mm.

Specifically, a number of the second through-holes 19 is not less than one, and the second through-holes 19 are arranged on a lower half of the second divider 14 at intervals, so that the liquid fragrance in the first cavity 15 can only pass through the lower half of the second divider 14. An upper half of the second divider 14 is used as a baffle, to block the liquid fragrance in the first cavity 15 to pass through the upper half of the second divider 14. Compared to a situation that the second through-holes 19 are distributed simultaneously in the upper half and the lower half of the second divider 14, this can avoid the liquid fragrance flowing into the upper half of the second divider 14 from obstructing the upward movement of gas from the lower half of the second divider 14. At the same time, compared to a situation that the second through-holes 19 are only distributed in the upper half of the second divider 14, it can provide space for the upward movement of the gas, avoid stagnation, and make the reflux smoother and more stable.

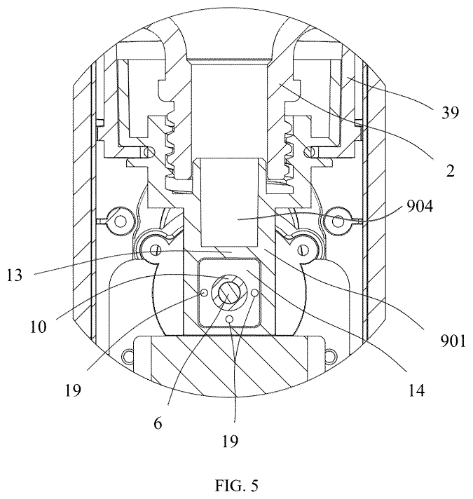

For example, the second through-hole 19 is one in quantity, and the second through-hole 19 is located at the lower half of the second divider 14; the second through-hole 19 is multiple in quantity, and the multiple second through-holes 19 are arranged on the lower half of the second divider 14 at intervals; or, as shown in FIG. 5, the second through-hole 19 is three in quantity, and one of the three second through-holes 19 is located at a middle position of a bottom of the second divider 14, and the other two of the three second through-holes 19 are located on a middle part of the second divider 14 and respectively located at two sides of the liquid-conducting core rod 6.

The number of the second through-holes 19 are determined according to atomization amount and a sectional area of the second divider 14. Due to a certain viscosity of the liquid fragrance, in order to ensure that the liquid fragrance can smoothly pass through the second through-hole 19, in an embodiment, a diameter of the second through-hole 19 is not less than 1 mm.

In another embodiment, a third divider 21 is disposed on a peripheral surface of the casing pipe 10 between the atomizing seat 9 and the second divider 14 and extending radially along the casing pipe 10. A peripheral side of the third divider 21 is connected to the inner wall of the atomizing seat 9. A cavity A 161 is defined between the third divider 21 and the second divider 14, a cavity B 162 is defined between the third divider 21 and the atomizing sheet 7, and the cavity A 161 is separated from the cavity B 162. The first divider 13 further defines a third through-hole 20, and the third through-hole 20 is configured to connect the upper cavity 17 and the cavity A 161.

By setting the third divider 21, the embodiment can seal the liquid fragrance flowing in from the first cavity 15 in the cavity A 161, and divide the atomizing sheet 7 and the cavity A 161, thereby avoiding the liquid fragrance in the second cavity 16 to directly contact with the atomizing sheet 7, resulting in a large amount of leakage. In an embodiment, the peripheral side of the third divider 21 is tightly fitted with the inner wall of the atomizing seat 9. In an embodiment, the casing pipe 10, the second divider 14 and the third divider 21 are an integrally formed component, which is convenient for processing and can also reduce assembly links, and a material of the integrally formed component is fluorine glue.

Based on the above fragrance diffuser, in an embodiment, the atomizing seat 9 includes a body 901 and a mist outlet head 902 detachably connected to the body 901. The casing pipe 10 is disposed in the body 901, and the atomizing sheet 7 is fixed between the body 901 and the mist outlet head 902. The mist outlet head 902 defines a mist outlet channel 903, an end of the mist outlet channel 903 is disposed facing towards the atomizing sheet 7, and another end of the mist outlet channel 903 is butted with the mist outlet 8. In the embodiment, the atomizing seat 9 includes the body 901 and the mist outlet head 902, the mist outlet head 902 is detachably connected to the body 901, which facilitates the installation of the casing pipe 10 and the atomizing sheet 7.

In an embodiment, a first groove 22 is defined between the body 901 and the mist outlet head 902 in annular. A scaling ring 23 is disposed in the first groove 22, the sealing ring 23 defines a second groove 24 in annular, and a peripheral edge of the atomizing sheet 7 is embedded in the second groove 24. By setting the sealing ring 23, the embodiment can seal the liquid fragrance accumulated in the cavity B 162 within the cavity B 162, and can also avoid leakage caused by the liquid fragrance flowing from the atomizing sheet 7 and the liquid fragrance accumulated in the atomizing seat 9. A material of the scaling ring 23 is silicone rubber.

In a specific embodiment, as shown in FIG. 6, the first groove 22 is an annular U-shaped groove, the scaling ring 23 is U-shaped and matched with the first groove 22, outer side surfaces of the sealing ring 23 are tightly fitted with corresponding surfaces of the first groove 22 respectively, that is, radial sections of the sealing ring 23 include a bottom side, a first side and a second side, and the second side is closer to the mist outlet head 902 compared to the first side. In an embodiment, in order to avoid influence of differences in flatness or processing errors on the scaling effect, the bottom side, the first side, and the second side are all sealed with a protruding structure to achieve a tight fit. Specifically, a first protruding part 25 is disposed on a surface of the body 901 facing towards the first side in annular, a second protruding part 26 is disposed on the bottom side of the scaling ring 23 in annular, and a third protruding part 27 is disposed on the second side of the scaling ring 23 in annular. The first protruding part 25, the second protruding part 26 and the third protruding part 27 are tightly fitted with corresponding combining surfaces respectively, to thereby achieve good sealing effect. Compared to using a structure with a larger flat surface for scaling and bonding, this can reduce a fitting area between the outer side surfaces of the sealing ring 23 and the corresponding surfaces of the first groove 22, and can achieve tight fit through a smaller fitting surface, thereby avoiding the reduction of the sealing effect due to the differences in flatness or the processing errors. At the same time, through this combination of protruding structure design, it is not only convenient for the sealing ring 23 and the body 901 to be integrally formed and processed separately, but also does not increase the difficulty of forming and processing the sealing ring 23 and the body 901.

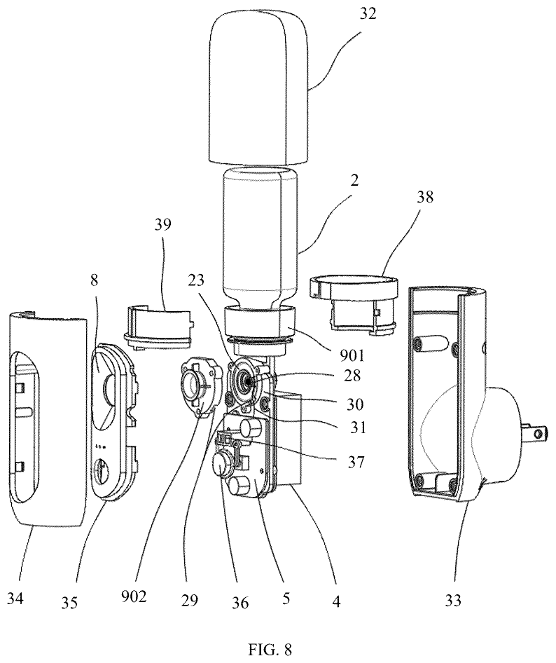

As shown in FIG. 8, the sealing ring 23 defines a first notch 28, the atomizing seat 9 defines a second notch 29, and a power cord 30 connected to the atomizing sheet 7 passes through the first notch 28 and the second notch 29 in sequence and winds out of the atomizing seat 9, so that the sealing performance of the sealing ring 23 is not damaged, contact between the liquid fragrance and the power cord 30 is avoid, thereby ensuring the sealing of the power cord 30 and meeting the routing requirements of the power cord 30.

In an embodiment, a base plate 31 is disposed in the housing 1, and a power supply 4 and a controller 5 are fixed on two sides of the base plate 31 in a detachable connection manner. The power supply 4 and the controller 5 are detachably connected to the base plate 31, and the power supply 4 is electrically connected to the controller 5. The atomizing sheet 7 is electrically connected to the controller 5, and the base plate 31 is fixedly connected to the body 901. In an embodiment, the base plate 31 and the body 901 are an integral structure, which can reduce a number of molds and assembly links, improve production efficiency. At the same time, two sides of the plate are used to fix the power supply 4 and the controller 5, which can improve the space utilization of the fragrance diffuser and reduce its external dimensions. Specifically, the materials of the base plate 31 and the body 901 are polyoxymethylene (POM).

In an embodiment, the housing 1 includes an upper housing 321, a lower housing and a connecting housing 60. The connecting housing 60 is fixedly sleeved on a periphery of the body 901, the upper housing 321 covers the bottle 2 and is clamped with an upper end of the connecting housing 60, so that when the bottle 2 needs to be replaced, it is only necessary to remove the upper housing 321 to implement the replacement, which is convenient and quick. The lower housing includes a first housing part 33, a second housing part 34 and a third housing part 35. The third housing part 35 defines the mist outlet 8, the third housing part 35 is matched with the controller 5, and the mist outlet 8 is butted with the mist outlet channel 903 of the mist outlet head 902 after the third housing part 35 is matched with the controller 5. In the embodiment, the controller 5 is provided with a control button 36 and a working status indicator lamp 37, and the third housing part 35 correspondingly defines through-holes. When the third housing part 35 is aligned with the controller 5, the control button 36 and the working status indicator lamp 37 are exposed from the corresponding through-holes of the third housing part 35. The first housing part 33 and the second housing part 34 are connected to cover the connecting housing 60, the power supply 4 and the controller 5 and clamp the third housing part 35. When installing, the third housing part 35 is aligned with the controller 5 firstly, then the first housing part 33 and the second housing part 34 are connected to complete installation of the lower housing. Specifically, the first housing part 33 and the second housing part 34 are also fixed by screws after connecting. When it is necessary to repair the parts in the lower housing, only the first housing part 33 and/or the second housing part 34 need to be disassembled to expose the parts, which is convenient for maintenance. The structural design of the housing 1 in the embodiment can take into account the convenient replacement of the bottle 2 and the convenient installation and maintenance of the electronic components, forming a completely wrapped housing structure, which is helpful for the aesthetic design of the appearance and improves the aesthetic appearance of the product.

It should be noted that the connecting housing 60 of the embodiment is further used to fix the first housing part 33 and the second housing part 34 vertically and radially, so as to prevent the first housing part 33 and the second housing part 34 from rotating and shaking up and down. In addition, as shown in FIG. 1 and FIG. 8, the connecting housing 60 includes a first casing 38 and a second casing 39, and the first casing 38 is spliced with the second casing 39. Specifically, the second casing 39 and the second housing part 34 are located at a same side. During installation, the first casing 38 and the second casing 39 can be fixedly casing piped on the periphery of the body 901 in sequence, and then the installation of the first housing part 33, the second housing part 34 and the third housing part 35 is performed. Compared to the integrated structure of the connecting housing 60, this spliced structure can reduce the installation difficulty of the connecting housing 60, which greatly facilitate the installation of the connecting housing 60.

The disclosure provides a fragrance diffuser, which includes a housing 1 and a bottle 2 defining an accommodation cavity 3 therein. An atomizing head assembly is disposed in the housing 1 and connected to the bottle 2. The atomizing head assembly includes a liquid-conducting core rod 6, an atomizing sheet 7, an atomizing seat 9, a casing pipe 10 and a pressure relief mechanism 40. The atomizing seat 9 is connected to the bottle 2, and defines a liquid accumulation space 904, and the liquid accumulation space 904 is located below the accommodation cavity 3. The accommodation cavity 3 is connected to the liquid accumulation space 904 through the pressure relief mechanism 40. The casing pipe 10 is located in the atomizing seat 9. The housing 1 defines a mist outlet 8 facing towards the atomizing sheet 7, and the casing pipe 10 is located on a side of the atomizing sheet 7 facing away from the mist outlet 8. The liquid-conducting core rod 6 is inserted into the casing pipe 10, a first end of the liquid-conducting core rod 6 is connected to the liquid accumulation space 904, and a second end of the liquid-conducting core rod 6 is connected to the atomizing sheet 7. Liquid fragrance in the liquid accumulation space 904 flows from the first end of the liquid-conducting core rod 6 to the second end of the liquid-conducting core rod 6, so that the liquid fragrance is atomized by the atomizing sheet 7, and then diffused to the air from the mist outlet 8.

In the fragrance diffuser of the disclosure, the atomizing seat 9 has the liquid accumulation space 904, the casing pipe 10 is disposed in the atomizing seat 9, the first end of the liquid-conducting core rod 6 is connected to the liquid accumulation space 904, and the second end of the liquid-conducting core rod 6 is abutted against the atomizing sheet 7. The liquid-conducting core rod 6 is inserted into the casing pipe 10, which can reduce liquid pressure on the surface of the liquid-conducting core rod 6, thereby reducing the liquid fragrance flowing to the second end of the liquid-conducting core rod 6, and avoiding excessive liquid fragrance flowing to the second end of the liquid guiding core rod 6, so that the liquid fragrance in the liquid accumulation space 904 is not easy to leak out of the mist outlet 8, thereby reducing leakage. The accommodation cavity 3 is connected to the liquid accumulation space 904 through the pressure relief mechanism 40. After the pressure relief mechanism 40 is used to release the pressure in the accommodation cavity 3, the liquid fragrance can be transported to the liquid accumulation space 904 to ensure that the hydraulic pressure of the liquid in the liquid accumulation space 904 decreases, thereby further ensuring that the liquid fragrance in the liquid accumulation space 904 is not easily leaked outside the mist outlet 8.

In an embodiment, a first divider 13 is disposed in the atomizing seat 9 and located above the casing pipe 10. The first divider 13 is configured to divide a pressure relief cavity 406 and a lower cavity in the atomizing seat 9, the pressure relief cavity 406 is connected to the lower cavity, and the casing pipe 10 and the liquid-conducting core rod 6 are located in the lower cavity. The pressure relief mechanism 40 is detachably installed in the pressure relief cavity 406, which is convenience for forming the pressure relief mechanism 40 and the pressure relief cavity 406 separately. The accommodation cavity 3 is connected to the pressure relief cavity 406 through the pressure relief mechanism 40, so that pressure of the liquid fragrance in the pressure relief cavity 406 is small.

In an embodiment, the pressure relief mechanism 40 defines a pressure relief hole 401 and a gas return hole 402, an input end and an output end of the gas return hole 402 are respectively connected to the pressure relief cavity 406 and the accommodation cavity 3, and an input end and an output end of the pressure relief hole 401 are respectively connected to the accommodation cavity 3 and the pressure relief cavity 406. The gas return hole 402 is used to return the gas into the accommodation cavity 3 to ensure pressure balance, the pressure relief hole 401 is configured to discharge the liquid fragrance in the accommodation cavity 3 into the pressure relief cavity 406. The pressure relief hole 401 generally has a smaller diameter, which can reduce the pressure of the liquid fragrance in the accommodation cavity 3 and then discharge the liquid fragrance into the pressure relief cavity 406, to thereby playing a role in pressure relief. The pressure relief mechanism 40 defines the pressure relief hole 401 and the gas return hole 402 thereon, so that the gas return and oil discharge in the accommodation cavity 3 are independent of each other and do not interfere with each other.

In an embodiment, the gas return hole 402 is higher than the pressure relief hole 401, which refers to that a height of the gas return hole 402 is higher than that of the pressure relief hole 401 in use state, the gas return hole 402 is deeper into the accommodation cavity 3 than the pressure relief hole 401, which is more conducive to the gas return and the discharge of the liquid fragrance in the accommodation cavity 3, and the accommodation cavity 3 is also easier to discharge the liquid fragrance. Due to a certain viscosity of the liquid fragrance, it is difficult for the liquid fragrance to pass through small holes, while the gas can easily pass through small holes. When a cross-section of the pressure relief hole 401 is greater than that of the gas return hole 402, it ensures that gas flows through the gas return hole 402, while the liquid fragrance flow through the pressure relief hole 401.

In an embodiment, the pressure relief mechanism 40 defines an oil accumulation groove 403 thereon, the oil accumulation groove 403 is located on a bottom of the accommodation cavity 3 and connected to the accommodation cavity 3. The pressure relief hole 401 is defined on a bottom of the oil accumulation groove 403, and the gas return hole 402 is higher than the oil accumulation groove 403.

In an embodiment, the output end of the pressure relief hole 401 defines a liquid-conducting groove 404 connected to the pressure relief hole 401, a sectional area of the liquid-conducting groove 404 is greater than that of the pressure relief hole 401, the liquid-conducting groove 404 is disposed extending along the pressure relief cavity 406, and an output end of the liquid-conducting groove 404 is connected to the pressure relief cavity 406.

In an embodiment, the input end of the gas return hole 402 defines a gas accumulation groove 405 connected to the gas return hole 402, a sectional area of the gas accumulation groove 405 is greater than that of the gas return hole 402, the gas accumulation groove 405 is disposed extending along the pressure relief cavity 406, and an input end of the gas accumulation groove 405 is connected to the pressure relief cavity 406.

In an embodiment, the first divider 13 further defines a first through-hole 18 and a third through-hole 20, the first through-hole 18 and the third through-hole 20 are respectively located on two sides of the second divider 14, and the first through-hole 18 and the third through-hole 20 are configured to connect the pressure relief cavity 406 and the second cavity 16. Positions of the first through-hole 18 and the third through-hole 20 are respectively configured to correspond to the pressure relief hole 401 and the gas return hole 402.

In an embodiment, an end of the pressure relief cavity 406 facing away from the first divider 13 defines an opening 408, an end of the pressure relief mechanism 40 is inserted into the opening 408, and the pressure relief mechanism 40 is in sealing fit with the opening 408. The pressure relief mechanism 40 includes a limit flange 407 at the opening 408, and the limit flange 407 protrudes outward from the opening 408. A bottom of the bottle 2 has a bottle mouth 409 connected to the accommodation cavity 3, the gas return hole 402 is defined on the bottle mouth 409 and extends along a direction of the accommodation cavity 3, and the pressure relief hole 401 is located below the bottle mouth 409. A diameter of the limit flange 407 is greater than a diameter of the opening 408 on the top of the pressure relief cavity 406.

The disclosure provides another embodiment of the fragrance diffuser.

The fragrance diffuser further includes a middle element 50, and the middle element 50 is disposed between the second end of the liquid-conducting core rod 6 and the atomizing sheet 7. A side of the middle element 50 is connected to the second end of the liquid-conducting core rod 6, and another side of the middle element 50 is connected to the atomizing sheet 7. The middle element 50 is configured to transport the liquid fragrance from the liquid-conducting core rod 6 to the atomizing sheet 7, and a concentration of the liquid fragrance on the atomizing sheet 7 is lower than a concentration of the liquid fragrance on the second end of the liquid-conducting core rod 6. Due to a low concentration of the liquid fragrance transported to the atomizing sheet 7, the liquid fragrance is not easy to be leaked from the atomizing sheet 7.

A connection area of the middle element 50 and the liquid-conducting core rod 6 is smaller than a connection area of the middle element 50 and the atomizing sheet 7. The middle element 50 is a liquid-conducting cotton sheet, and a cross-section of the liquid-conducting cotton sheet is greater than that of the liquid-conducting core rod 6. When the liquid-conducting core rod 6 is subjected to a certain pressure, its ability to output liquid fragrance is constant. However, when the liquid-conducting core rod 6 is directly connected to the atomizing sheet 7, the concentration of the liquid fragrance at the connection position between the liquid-conducting core rod 6 and the atomizing sheet 7 is high, and the liquid fragrance is prone to leak out of the mist outlet 8. The cross-section of the liquid-conducting cotton sheet is greater than that of the liquid-conducting core rod 6, resulting in a lower concentration of the liquid fragrance output from the liquid-conducting core rod 6 to the liquid-conducting cotton sheet.

In the specification, unless otherwise specified and limited, the first feature “above” or “below” the second feature can be a direct contact between the first feature and the second feature, or an indirect contact between the first feature and the second feature through an intermediate medium. Moreover, the first feature being “above” the second feature may indicate that the first feature is directly above or diagonally above the second feature, or simply that the first feature is horizontally higher than the second feature. The first feature “below” the second feature may indicate that the first feature is directly below or diagonally below the second feature, or simply indicate that the first feature has a lower horizontal height than the second feature.

In the description of the specification, the reference to terms such as “embodiments”, “still another embodiment”, “other embodiments”, or “specific examples” mean that the specific features, structures, materials, or characteristics described in conjunction with the embodiments or examples are included in at least one embodiment or example of the disclosure. In the specification, the schematic expressions of the above terms do not necessarily refer to the same embodiments or examples. Moreover, the specific features, structures, materials, or characteristics described can be combined in any one or more embodiments or examples in an appropriate manner. In addition, those skilled in the art can combine the different embodiments or examples described in the specification, as well as the features of different embodiments or examples, without conflicting with each other.

Although the embodiments of the disclosure have been shown and described above, it can be understood that the above embodiments are exemplary and should not be construed as limiting the disclosure. Those skilled in the art may make changes, modifications, substitutions, and variations to the above embodiments within the scope of the disclosure.

What is claimed is:

1. A fragrance diffuser, comprising a housing and a bottle defining an accommodation cavity therein, wherein an atomizing head assembly is disposed in the housing and connected to the bottle; the atomizing head assembly comprises a liquid-conducting core rod, an atomizing sheet, an atomizing seat and a casing pipe; the atomizing seat is connected to the bottle and defines a liquid accumulation space therein, and the liquid accumulation space is connected to the accommodation cavity and located below the accommodation cavity; the casing pipe is located in the atomizing seat, the housing defines a mist outlet facing towards the atomizing sheet, and the casing pipe is located on a side of the atomizing sheet facing away from the mist outlet; the liquid-conducting core rod is inserted into the casing pipe, a first end of the liquid-conducting core rod is connected to the liquid accumulation space, a second end of the liquid-conducting core rod is abutted against the atomizing sheet, and the liquid-conducting core rod is configured to flow liquid fragrance in the liquid accumulation space from the first end of the liquid-conducting core rod to the second end of the liquid-conducting core rod.

2. The fragrance diffuser as claimed in claim 1, wherein a first divider is disposed in the atomizing seat and located above the sleeve, the first divider is configured to divide an upper cavity and a lower cavity in the atomizing seat, the upper cavity is connected to the lower cavity, and the casing pipe and the liquid-conducting core rod are located in the lower cavity.

3. The fragrance diffuser as claimed in claim 2, wherein a second divider is disposed on a peripheral surface of the casing pipe and extending radially along the casing pipe, a peripheral side of the second divider is connected to the first divider and an inner wall of the atomizing seat to divide the lower cavity into a first cavity and a second cavity, the first divider defines a first through-hole, the second divider defines a second through-hole, the first through-hole is configured to connect the upper cavity and the lower cavity, and the second through-hole is configured to connect the first cavity and the second cavity.

4. The fragrance diffuser as claimed in claim 3, wherein the first divider further defines a third through-hole, the first through-hole and the third through-hole are respectively located at two sides of the second divider, and the third through-hole is configured to connect the upper cavity and the second cavity.

5. The fragrance diffuser as claimed in claim 3, wherein the second through-hole is one in quantity, and the second through-hole is located at a lower half of the second divider;
the second through-hole is multiple in quantity, and the multiple second through-holes are arranged on the lower half of the second divider at intervals; or
the second through-hole is three in quantity, and one of the three second through-holes is located at a middle position of a bottom of the second divider, and the other two of the three second through-holes are located on a middle part of the second divider and respectively located at two sides of the liquid-conducting core rod.

6. The fragrance diffuser as claimed in claim 4, wherein a diameter of the second through-hole is not less than 1 mm, and a diameter of the third through-hole is not less than 1.5 mm.

7. The fragrance diffuser as claimed in claim 3, wherein a third divider is disposed on a peripheral surface of the casing pipe between the atomizing seat and the second divider and extending radially along the casing pipe, a peripheral side of the third divider is connected to the inner wall of the atomizing seat to define a cavity A between the third divider and the second divider and define a cavity B between the third divider and the atomizing sheet, the cavity A is separated from the cavity B, the first divider further defines a third through-hole, and the third through-hole is configured to connect the upper cavity and the cavity A.

8. The fragrance diffuser as claimed in claim 1, wherein the atomizing seat comprises a body and a mist outlet head detachably connected to the body, the casing pipe is disposed in the body, the atomizing sheet is fixed between the body and the mist outlet head, the mist outlet head defines a mist outlet channel, an end of the mist outlet channel is disposed facing towards the atomizing sheet, and another end of the mist outlet channel is butted with the mist outlet.

9. The fragrance diffuser as claimed in claim 8, wherein a first groove is defined between the body and the mist outlet in an annular configuration, a sealing ring is disposed in the first groove, the sealing ring defines a second groove, and a peripheral edge of the atomizing sheet is embedded in the second groove.

10. The fragrance diffuser as claimed in claim 9, wherein the first groove is an annular U-shaped groove, the sealing ring is U-shaped and matched with the first groove, and outer side surfaces of the sealing ring are configured to tightly fit with corresponding surfaces of the first groove respectively; and the sealing ring defines a first notch, the atomizing seat defines a second notch, and a power cord connected to the atomizing sheet sequentially passes through the first notch and the second notch and winds out of the atomizing seat.

11. The fragrance diffuser as claimed in claim 9, wherein a base plate is disposed in the housing, a power supply is disposed on a side of the base plate, a controller is disposed on another side of the base plate, the power supply and the controller are detachably connected to the base plate, the power supply is electrically connected to the controller, the atomizing sheet is electrically connected to the controller, and the base plate is connected to the body.

12. The fragrance diffuser as claimed in claim 11, wherein the housing comprises an upper housing, a lower housing and a connecting housing, the connecting housing is fixedly sleeved on a periphery of the body, and the upper housing covers the bottle and is clamped with an upper end of the connecting housing;
wherein the lower housing comprises a first housing part, a second housing part and a third housing part, the third housing part defines the mist outlet, the third housing part is matched with the controller, and the mist outlet is butted with the mist outlet channel of the mist outlet head after the third housing part is matched with the controller; and
wherein the first housing part and the second housing part are spliced and connected to cover the connecting housing, the power supply and the controller and fixedly clamp the third housing part.

13. The fragrance diffuser as claimed in claim 1, wherein the atomizing head assembly further comprises a pressure relief mechanism, and the accommodation cavity is connected to the liquid accumulation space through the pressure relief mechanism.

14. The fragrance diffuser as claimed in claim 13, wherein a first divider is disposed in the atomizing seat and located above the casing pipe, the first divider is configured to divide a pressure relief cavity and a lower cavity in the atomizing seat, the pressure relief cavity is connected to the lower cavity, and the sleeve and the liquid-conducting core rod are located in the lower cavity; and the pressure relief mechanism is detachably installed in the pressure relief cavity, and the accommodation cavity is connected to the pressure relief cavity through the pressure relief mechanism.

15. The fragrance diffuser as claimed in claim 14, wherein the pressure relief mechanism defines a pressure relief hole and a gas return hole, an input end and an output end of the gas return hole are respectively connected to the pressure relief cavity and the accommodation cavity, and an input end and an output end of the pressure relief hole are respectively connected to the accommodation cavity and the pressure relief cavity.

16. The fragrance diffuser as claimed in claim 15, wherein the gas return hole is higher than the pressure relief hole, and a cross-section of the pressure relief hole is greater than that of the gas return hole;
wherein the pressure relief mechanism defines an oil accumulation groove, the oil accumulation groove is located on a bottom of the accommodation cavity and connected to the accommodation cavity, the pressure relief hole is defined on a bottom of the oil accumulation groove, and the gas return hole is higher than the oil accumulation groove;
wherein the output end of the pressure relief hole defines a liquid-conducting groove connected to the pressure relief hole, a sectional area of the liquid-conducting groove is greater than that of the pressure relief hole, the liquid-conducting groove is disposed extending along the pressure relief cavity, and an output end of the liquid-conducting groove is connected to the pressure relief cavity; and
wherein the input end of the gas return hole defines a gas accumulation groove connected to the gas return hole, a sectional area of the gas accumulation groove is greater than that of the gas return hole, the gas accumulation groove is disposed extending along the pressure relief cavity, and an input end of the gas accumulation groove is connected to the pressure relief cavity.

17. The fragrance diffuser as claimed in claim 15, wherein the first divider further defines a first through-hole and a third through-hole, the first through-hole and the third through-hole are respectively located on two sides of the second divider, and the first through-hole and the third through-hole are configured to connect the pressure relief cavity and a second cavity; and positions of the first through-hole and the third through-hole are respectively configured to correspond to the pressure relief hole and the gas return hole; and
wherein an end of the pressure relief cavity facing away from the first divider defines an opening, an end of the pressure relief mechanism is inserted into the opening, and the pressure relief mechanism is in sealing fit with the opening; the pressure relief mechanism comprises a limit flange at the opening, and the limit flange protrudes outward from the opening; and a bottom of the bottle has a bottle mouth connected to the accommodation cavity, the gas return hole is defined on the bottle mouth and extends along a direction of the accommodation cavity, and the pressure relief hole is located below the bottle mouth.

18. The fragrance diffuser as claimed in claim 1, wherein the atomizing head assembly further comprises a middle element, the middle element is disposed between the second end of the liquid-conducting core rod and the atomizing sheet, a side of the middle element is connected to the second end of the liquid-conducting core rod, and another side of the middle element is connected to the atomizing sheet; and the middle element is configured to transport the liquid fragrance from the second end of the liquid-conducting core rod to the atomizing sheet, a concentration of the liquid fragrance on the atomizing sheet is lower than a concentration of the liquid fragrance on the second end of the liquid-conducting core rod, and the atomizing sheet is configured to atomize the liquid fragrance on the middle element and disperse atomized liquid fragrance into air from the mist outlet.

19. The fragrance diffuser as claimed in claim 18, wherein a connection area of the middle element and the liquid-conducting core rod is smaller than that of the middle element and the atomizing sheet; and the middle element is a liquid-conducting cotton sheet.
